(19) United States
(12) Patent Application Publication
(10) Pub. No.: US 2018/0008297 A1
(43) Pub. Date: Jan. 11, 2018

(54) PRESSURE WAVE TRANSDUCER

(71) Applicant: Moshe Ein-Gal, Hasharon (IL)

(72) Inventor: Moshe Ein-Gal, Hasharon (IL)

(21) Appl. No.: 15/206,440

(22) Filed: Jul. 11, 2016

(51) Int. Cl. A61B 17/225; A61N 7/00

(52) U.S. Cl. CPC A61B 17/2251; A61N 2007/0078

(57) ABSTRACT

A system and method for producing pressure waves accelerating a projectile membrane in response to a pulse of force produced by a power source. The accelerating membrane delivers kinetic energy to a target membrane upon collision. Following the collision, the power source may continue to deliver additional energy to the now-in-contact membranes. The kinetic energy of the impact and the additional energy following the impact contribute to producing pressure waves by the target membrane.

### FIELD OF THE INVENTION

The present invention relates to a method and a system for shockwave generation and shockwave treatment, wherein the shockwave generator (transducer) is in direct contact with a part of the patient's body without using a propagating liquid.

### BACKGROUND OF THE INVENTION

In many prior art systems for ESWL (extracorporeal shockwaves lithotripsy) there is a thin metallic membrane, which is configured to jerk upon being subjected to a short pulse of an inductive electromagnetic force. The pulse length is determined by the electrical circuit used for the pulse generation. The jerking membrane, typically in contact with propagation liquid, produces a pressure wave in the liquid. The wave front, initially parallel to the membrane, can be modified by reflectors, lenses, etc., prior to coupling to a patient. Due to non-linear acoustic properties of the propagation liquid, the wave front gets steeper and eventually the wave may turn into a shockwave. The fraction of the acoustic energy delivered to the liquid by the membrane is determined by the respective acoustic impedances of the membrane and the liquid.

A variation of the electromagnetic method is described in US application 2015/0231414 to Ein-Gal. In this method, a solid non-metallic, coupling interface replaces the propagating liquid of the electromagnetic method. The interface, tightly attached to the metallic membrane on one side and to a patient on the other side, directly delivers the waves produced by the metallic membrane to the patient. The fraction of the acoustic energy delivered to the interface by the membrane is determined by the respective acoustic impedances of the membrane and the interface.

### SUMMARY OF THE INVENTION

The present invention seeks to provide a novel pressure wave system and method, as is described more in detail hereinbelow, which has use in many medical applications, such as but not limited to, lithotripsy, orthopedics, treating pathological tissue conditions and many others, in particular, applications to soft tissue.

In one embodiment, pressure waves are created by accelerating a projectile membrane in response to a pulse of force produced by a power source. The accelerating membrane delivers kinetic energy to a target membrane upon collision. Following the collision, the power source may continue to deliver additional energy to the now-in-contact membranes. The kinetic energy of the impact and the additional energy following the impact contribute to producing pressure waves by the target membrane

There is thus provided in accordance with a non-limiting embodiment of the invention, a system including a projectile membrane and a target membrane initially spaced from the projectile membrane by a gap, and a power source operative to produce a force to accelerate the projectile membrane towards the target membrane, the force causing the projectile membrane to travel over the gap and impact the target membrane with an impact force that transfers kinetic energy to the target membrane and creates a pressure pulse at the target membrane, the target membrane being configured to deliver acoustic pressure pulses to an object.

The target membrane may or may not contact the object. A biasing device may be configured to move the projectile membrane back to its initial position.

In accordance with a non-limiting embodiment of the invention an array is provided of more than one projectile membrane and more than one target membrane.

In accordance with a non-limiting embodiment of the invention a sequencer is incorporated for sequencing the energy delivered to the more than one projectile membrane.

There is also provided in accordance with a non-limiting embodiment of the invention a method including providing at least one transducer, each transducer including a projectile membrane and a target membrane initially spaced from the projectile membrane by a gap, and a power source operative to produce a force to accelerate the projectile membrane towards the target membrane, the force causing the projectile membrane to travel over the gap and impact the target membrane with an impact force that transfers kinetic energy to the target membrane and creates a pressure pulse at the target membrane, the target membrane being configured to deliver acoustic pressure pulses to an object. The method may further include using the at least one transducer to deliver acoustic pressure pulses to the object.

In accordance with a non-limiting embodiment of the invention a time duration of the force applied to the projectile membrane is longer than a time required for the projectile membrane to reach the target membrane.

In accordance with a non-limiting embodiment of the invention a remainder of the force which is still applied to the projectile membrane following the impact creates an additional pressure on the projectile membrane, and the additional pressure is also transferred to the target membrane which is already in contact with the projectile membrane.

The method may include modifying pressure on the target membrane by modifying materials of the membranes, modifying a magnitude of the initial gap between the membranes, or modifying a force pulse produced by the power source.

### BRIEF DESCRIPTION OF THE DRAWINGS

The present invention will be understood and appreciated more fully from the following detailed description taken in conjunction with the drawings in which:

FIGS. 1 and 2 are simplified illustrations of a system (transducer) for pressure wave generation or treatment, constructed and operative in accordance with an embodiment of the invention, showing a projectile membrane prior to and after impacting a target membrane, respectively.

FIG. 3 is a simplified illustration of an array of such transducers, constructed and operative in accordance with another embodiment of the invention.

### DETAILED DESCRIPTION OF EMBODIMENTS

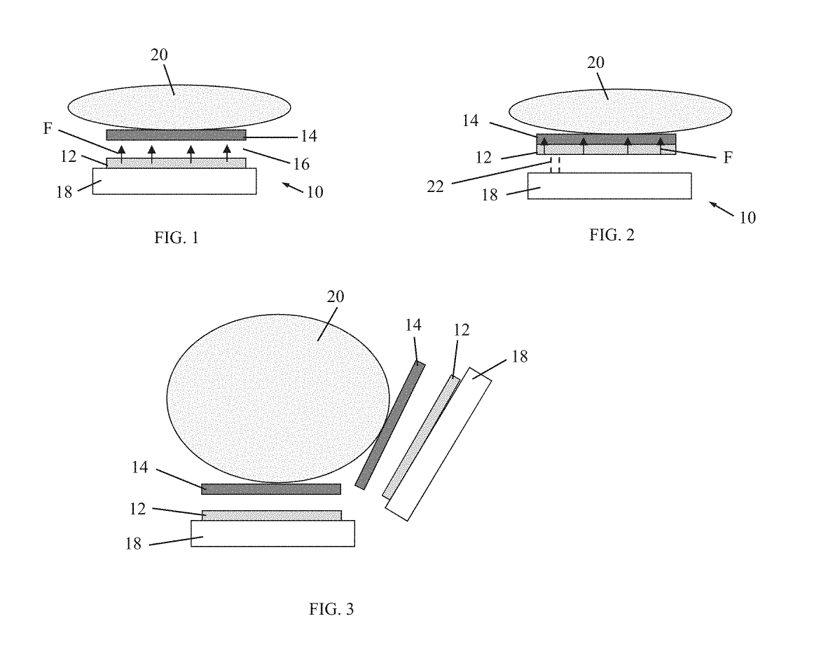

Reference is now made to FIG. 1, which illustrates a system 10 for producing acoustic pressure pulses or shockwaves, constructed and operative in accordance with a non-limiting embodiment of the invention.

System (also referred to as transducer) 10 includes a projectile membrane 12 and a target membrane 14 initially spaced from projectile membrane 12 by a gap 16 (such as but not limited to, 2 mm or less). A power source 18 is operative to produce a force F to accelerate projectile membrane 12 towards target membrane 14. The force causes projectile membrane 12 to travel over gap 16 and impact target membrane 14 with an impact force that transfers kinetic energy to target membrane 14 and creates a pressure pulse at target membrane 14. Target membrane 14 is operable to deliver acoustic pressure pulses to an object 20, such as tissue. In one embodiment, target membrane 14 contacts object 20; in other embodiments, target membrane 14 does not necessarily contact object 20 (in which case, the wave front can be modified by reflectors, lenses, etc., prior to coupling to the patient).

The power source 18 may include, without limitation, electromagnetic, pneumatic, piezoelectric, electrohydraulic, ballistic, pyrotechnic and others. For an inductive electromagnetic power source, the projectile membrane 12 may be electrically conductive so that the electromagnetic power source induces a magnetic force that repels projectile membrane 12 (the target membrane may be non-conductive).

Projectile membrane 12 may move back to its initial position simply by gravitation or by means of a biasing device 22, such as but not limited to, a spring, damper, electromagnetic device (e.g., the electromagnetic power source is switched to attract the membrane 12), or pneumatic device.

The membranes may be of any size or shape, such as but not limited to, circular, rectangular, square, or polygonal or others and any combination thereof. The membranes may be planar, concave or convex and any combination thereof.

In one embodiment, the time duration of the force applied to projectile membrane 12 may be longer than the time required for the projectile membrane 12 to reach the target membrane 14. In such a case, the remaining part of the force which is still applied to the projectile membrane 12 following the impact creates an additional pressure on projectile membrane 12, which pressure is transferred to the target membrane 14 which is already in contact with the projectile membrane 12. The resulting pressure on the target membrane 14 is a combination of the ballistic component of the projectile membrane 12 up to contact with (impact on) the target membrane 12 the additional component due to the energy applied to the projectile membrane 12 during the time after the initial impact.

The magnitude of the ballistic component is a function, inter alia, of the coefficient of restitution of the materials of both membranes (that is, relative speed after impact divided by relative speed before impact). The additional component is a function, inter alia, of the acoustic impedances of both membranes. The combined pressure on the target membrane 14 may be optimized or modified by selecting the membrane materials, selecting the magnitude of the initial gap between the membranes, selecting the shape of the force pulse produced by the power source, and other factors.

The system may include more than one such transducer arranged in any suitable array. The transducer array may be arranged to produce combined waves that are converging, diverging or planar. One power source may be used or a plurality of power sources, such as one power source dedicated to each projectile membrane. The power source or sources may energize the transducers of the array at different times to produce different pressure waves and effects. The transducers of the array may be movable with respect to each other to produce different combined pressure waves and effects.

What is claimed is:

1. A system comprising:
a projectile membrane and a target membrane initially spaced from said projectile membrane by a gap; and
a power source operative to produce a force to accelerate said projectile membrane towards said target membrane, the force causing said projectile membrane to travel over said gap and impact said target membrane with an impact force that transfers kinetic energy to said target membrane and to said target membrane, said target membrane being configured to deliver acoustic pressure pulses to an object.

2. The system according to claim 1, wherein said target membrane contacts said object.

3. The system according to claim 1, wherein said target membrane does not contact said object.

4. The system according to claim 1, further comprising a biasing device configured to move said projectile membrane back to its initial position.

5. The system according to claim 1, further comprising an array of more than one said projectile membrane and more than one said target membrane.

6. The system according to claim 5, further incorporating a sequencer operable to sequence force pulses corresponding to the more than one said projectile membrane.

7. The system according to claim 1, wherein at least one said power source produces the force to accelerate said projectile membranes towards said target membranes.

8. A method comprising:
providing at least one transducer, each transducer comprising a projectile membrane and a target membrane initially spaced from said projectile membrane by a gap, and a power source operative to produce a force to accelerate said projectile membrane towards said target membrane, the force causing said projectile membrane to travel over said gap and impact said target membrane with an impact force that transfers kinetic energy to said target membrane and creates a pressure pulse at said target membrane, said target membrane being configured to deliver acoustic pressure pulses to an object.

9. The method according to claim 8, comprising using said at least one transducer to deliver acoustic pressure pulses to the object.

10. The method according to claim 8, wherein a time duration of the force applied to said projectile membrane is longer than a time required for said projectile membrane to reach said target membrane.

11. The method according to claim 10, wherein a remainder of the force which is still applied to said projectile membrane following the impact creates an additional pressure on said projectile membrane, and said additional pressure is transferred to said target membrane which is already in contact with said projectile membrane.

12. The method according to claim 10, comprising modifying pressure on said target membrane by modifying materials of said membranes.

13. The method according to claim 10, comprising modifying pressure on said target membrane by modifying a magnitude of the initial gap between the membranes.

14. The method according to claim 10, comprising modifying pressure on said target membrane by modifying a force pulse produced by the power source.

15. The method according to claim 8, comprising using said more than one transducer arranged in an array to deliver acoustic pressure pulses to the object.

16. The method according to claim 15, comprising using said array to deliver converging acoustic pressure pulses to the object.

17. The method according to claim 15, comprising using said array to deliver diverging acoustic pressure pulses to the object.

18. The method according to claim 15, comprising using said array to deliver planar acoustic pressure pulses to the object.

19. The method according to claim 15, comprising moving at least one of said transducers of said array with respect to another one of said transducers.
